(19) United States
(12) Patent Application Publication
(10) Pub. No.: US 2021/0402046 A1
(43) Pub. Date: Dec. 30, 2021

(54) DISINFECTING SANITARY SYSTEM FOR INACTIVATING AIRBORNE PATHOGENS WITHIN A SANITARY DEVICE

(71) Applicant: City University of Hong Kong, Hong Kong (HK)

(72) Inventor: Chi Keung LAI, Hong Kong (HK)

(21) Appl. No.: 17/356,626

(22) Filed: Jun. 24, 2021

Related U.S. Application Data
(60) Provisional application No. 63/044,480, filed on Jun. 26, 2020.

(51) Int. Cl. A61L 9/20

(52) U.S. Cl. CPC A61L 9/20; A61L 2209/11; A61L 2209/12

(57) ABSTRACT

A new disinfecting sanitary system utilizing an UV-C LED irradiation source is developed for disinfection of pathogens generated by toilet flushing. The disinfecting sanitary system includes a plurality of disinfection devices mounted on a hollow member of the sanitary device, and each of the disinfection devices is configured to emit a beam for disinfection; a control circuit coupled to the plurality of disinfection devices. Each of the plurality of disinfection devices including an ultraviolet light (UV) radiation apparatus configured to project UV radiation towards a target area for disinfection, and the control circuit controls the plurality of disinfection devices to project the UV radiation.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS-REFERENCE TO RELATED APPLICATIONS

The present application claims priority from U.S. Provisional Patent Application No. 63/044,480 filed Jun. 26, 2020, and the disclosure of which is incorporated herein by reference in its entirety.

### FIELD OF THE INVENTION

The present invention generally relates to the field of sanitary devices. More specifically, the present invention relates to a disinfecting sanitary system for a sanitary device.

### BACKGROUND OF THE INVENTION

Poor sanitation is one of the leading causative factors of infectious diseases such as cholera, diarrhea, dysentery, hepatitis A, typhoid and polio. Being an important facility for sanitation, the purpose of toilets is to provide a sanitation fixture for storage or disposal of human waste, including feces and urine, to improve hygienic conditions. However, the toilet and its immediate environment are recognized to be sources of bio-contamination and diverse types of bacteria have been detected in public restrooms. Toilet hygiene is not only an indoor air quality issue, but also a global issue and it has existed ever since it was first invented. Interestingly, not only the least developed countries have very poor hygiene environments in toilets, but also in developed countries, the risk of airborne pathogenic infection in toilets has been identified.

“Toilet plume” has been identified as a major contributor to the transmission of gastroenteric diseases. When flushing the toilet, toilet water can be atomized and forms copious pathogen-laden aerosol droplets. Depending on toilet design and other environmental factors such as flushing pressure, a single toilet flushing generates between hundreds of thousands and millions of potentially infectious aerosols. This in turn results in two routes of exposure or transmission of infectious airborne pathogens, namely inhalation and contact modes.

For the airborne pathway, infections occur by direct inhalation of pathogenic airborne droplets. Certain enteric pathogens, such as norovirus and enterohemorrhagic Escherichia coli (EHEC), can cause infections in low doses (less than 50 cells) with high probability of transmission. Even in a toilet immediately after flushing, the number of bacteria (e.g. Escherichia coli, Staphylococcus aureus, S. marcescens, Clostridium difficile, etc.) on the inner wall of the toilet can still be as high as one hundred thousand. Further, the bioaerosols can be detected even at a few tens of centimeters above the toilet seat persisting up to an hour after flushing. In contact mode infections, the fine and coarse pathogen-laden droplets can lead to surface or fomite contamination. Thus, rapidly falling fecal microbes cause microbial contamination of washroom surfaces, including doors, toilet seats, sinks, and floors. It is inevitable that a toilet user touches various surfaces inside the cubicle, as such contact exposures are no doubt are important risks, as toilet users may become infected whenever they touch surfaces that are already contaminated. This source of contamination is a major public health concern because hand contact with contaminated surfaces can result in self-inoculation through touching of the eyes, nose, or mouth. Therefore, toilet hygiene is a global issue. Finding an effective method to disinfect and sterilize sanitary facilities and prevent infectious diseases and cross-infection is a top priority.

It is reasonable and effective to control exposure at the precise location of emission if conditions permit. That is the concept of localized disinfection. Various commercial products have been developed, such as toilet seat papers, toilet seat disinfectant gels/foams and toilet bowl cleaners, which are useful to disinfect pre-existing contaminants on the toilet seats which can reduce the transmission of infections by contact mode. However, the actual sterilization effect of this method is relatively general, and it cannot directly kill all pathogens. Traditionally, medical practitioners focus more on the contact modes of transmission while less attention is paid to the airborne route. At present, toilets in most public restrooms are not equipped with disinfection devices. And it is often necessary to manually scrub with disinfectant or disinfection tablets to achieve the purpose of disinfection. This way may be effective for controlling the contact-based infection. However, none of these measures can completely prevent transmission through the aerosolization of fecal matter during toilet flushing.

Ultraviolet disinfection technology uses a high-efficiency, high-intensity, and long-life C-band ultraviolet (UV) light generating device to produce strong UV-C light to irradiate flowing water, air, and/or wall surfaces of a toilet. When various bacteria, viruses, parasites and other pathogens are irradiated with a certain dose of UV-C light, the DNA structures in their cells are destroyed, thereby killed without using any chemical, thereby achieving the purpose of disinfection and purification.

Recently, wall and ceiling-mounted disinfection units are becoming more popular in commercial buildings. Most of them utilize UV or ozone ions for the disinfection of pathogens. However, the devices are most often mounted at around the washing basins, which means that the disinfection actions would not take place until the pathogens are already well-mixed in the restroom. To date, no study has reported the quantitative disinfection performance of these devices in field settings. Therefore, in view of the shortcomings of the existing toilets, there is a need in the art to provide a new toilet with a safe and portable disinfection system that can more effectively disinfect and kill airborne and settled pathogens.

### SUMMARY OF THE INVENTION

One objective of the present invention is to provide a disinfecting sanitary system for toilets to address the above-mentioned shortcomings.

In accordance to one aspect of the present invention, the present invention provides a disinfecting sanitary system for inactivating airborne pathogens within a sanitary device. The disinfecting sanitary system includes a plurality of disinfection devices mounted on a hollow member of the sanitary device, and each of the disinfection devices is configured to emit a beam for disinfection; a control circuit coupled to the plurality of disinfection devices. Each of the plurality of disinfection devices including an ultraviolet light (UV) radiation apparatus configured to project UV radiation towards a target area for disinfection, and the control circuit controls the plurality of disinfection devices to project the UV radiation.

In accordance to one embodiment, the plurality of disinfection devices further includes an aluminum plate and a printed circuit board (PCB) fixed onto the aluminum plate.

In accordance to another embodiment, the plurality of disinfection devices further includes an input-output interface having a positive pole and a negative pole communicating with the UV radiation apparatus, wherein the input-output interface is connected to the control circuit via wires.

In accordance to one embodiment, the disinfecting sanitary system further includes a protection member wrapping the UV radiation apparatus.

In accordance to another embodiment, the protection member is a transparent film with an approximate thickness in the range of 0.1 to 0.2 mm.

In accordance to one embodiment, the UV radiation apparatus includes at least one light emitting diode (LED) distributed according to positions of a plurality of nozzles of the sanitary device, and the at least one LED is/are arranged to irradiate within UV-C band.

In accordance to another embodiment, the peak wavelength of the at least one LED is in the range of 100 nm to 280 nm.

In accordance to one embodiment, the plurality of disinfection devices surrounds a central axis of the hollow member at equal intervals.

In accordance to one embodiment, an opening of the hollow member comprising at least one region, and the plurality of disinfection devices are arranged in one or more of the regions.

In accordance to another embodiment, one or more of the plurality of disinfection devices are arranged at a first interval in a first region of the at least one region, wherein one or more of the plurality of disinfection devices are arranged at a second interval in a second region of the at least one region, wherein one or more of the plurality of disinfection devices are arranged at a third interval in a third region of the at least one region, and wherein the first interval, the second interval and the third interval are different.

In accordance to yet another embodiment, one or more of the plurality of disinfection devices are arranged in one region of the at least one region, and another region of the at least one region is not provided with the plurality of disinfection devices.

In accordance to one embodiment, the disinfecting sanitary system inactivates the airborne pathogens within a vertical distance of 0.4 m to 1.3 m from a ground floor level.

In accordance to one embodiment, the airborne pathogens are bioaerosols with a size of less than 0.3 μm, and the bioaerosols include airborne microorganisms or parasites.

In accordance to another embodiment, the microorganisms are select from the group consisting of Escherichia coli, Salmonella typhimurium, Staphylococcus epidermidis, Shigella dysenteriae, Listeria monocytogenes, Clostridium difficile, and Candida albicans.

In accordance to another embodiment, the parasites include Cryptosporidium.

In accordance to second aspect of the present invention, the present invention provides a sanitary device for inactivating airborne pathogens comprising the disinfecting sanitary system described in any one of the preceding embodiments.

In accordance to one embodiment, the sanitary device includes a container for receiving fluids and the airborne pathogens, and a hollow member positioned on the container.

In accordance to another embodiment, the sanitary device further contains an opening at least partly defined by the container.

In accordance to yet another embodiment, the hollow member is an aluminum ring, and the hollow member at least partly defining the opening.

Various embodiments of the present invention utilize an ultraviolet light (UV)-generating device to generate strong UV-C light to irradiate flowing water, air, and/or object surfaces, so that the DNA structures in cells of various bacteria, viruses, parasites and other pathogens are exposed to a certain dose of UV-C light and irradiated and destroyed, thereby killed without using any chemical, achieving the purpose of disinfection and purification. The disinfecting sanitary system of the present invention combines UV disinfection technology with the toilet, which can effectively kill reduce the breeding of bacteria and viruses after each toilet use, and can effectively prevent the spreading of contagious diseases caused by multiple users using the same toilet.

### BRIEF DESCRIPTION OF THE DRAWINGS

Embodiments of the invention are described in more details hereinafter with reference to the drawings, in which:

FIG. 1A depicts a schematic view of components of a disinfection device in accordance with one embodiment of the present invention.

FIG. 1B depicts a schematic view of components of a disinfection device in accordance with another embodiment of the present invention.

FIG. 2A depicts a schematic view of uniformly configured disinfection devices with 3-LEDs on the ring in accordance with one embodiment of the present invention.

FIG. 2B depicts a schematic view of uniformly configured disinfection devices with 5-LEDs on the ring in accordance with one embodiment of the present invention.

FIG. 2C depicts a schematic view of uniformly configured disinfection devices with 8-LEDs on the ring in accordance with one embodiment of the present invention.

FIG. 2D depicts a schematic view of concentratedly configured disinfection devices with 5-LEDs on the ring in accordance with one embodiment of the present invention.

FIG. 3 depicts a schematic view of uniformly configured disinfection devices with 10-LEDs on the ring in accordance with one embodiment of the present invention.

FIG. 4 depicts a schematic view of a position of the sensing probe for measuring UV irradiance in a toilet bowl.

FIG. 5 shows the relationship between mean LED irradiance and distance from the source.

FIG. 6 shows the efficacy of localized UV-C LEDs for surface disinfection.

FIG. 7 shows the efficacy of localized UV-C LEDs for airborne disinfection.

FIG. 8 shows a comparison between the disinfection efficacy of uniformly configured UV-C LEDs and two-sided UV-C LEDs.

### DETAILED DESCRIPTION

Toilets are potential sources for the transmission of fecal-borne diseases. Pathogens can survive for long periods of time both on toilet surfaces and in the air, making the entire environment a continuous reservoir of infectious agents. The risk of contracting diseases is even higher where toilets are shared among multiple users. Unfortunately, almost all of the well-known cleaning and disinfection approaches are done after a period of multiple uses. The fact that pathogens would not be removed after each toilet use creates a critical microbiological problem within the toilet environment. Besides the detrimental effects of inhaling polluted air on the wellbeing of users, contact of the human body with toilet surfaces, even though not likely to facilitate infection, can also promote the transfer of microorganisms between persons. Another major concern is the formation of biofilms under favorable conditions following the adhesion of pathogens to toilet surfaces. The control of toilet infections must, therefore, involve the disinfection of both air and surfaces in the toilet microenvironment.

In the following description, the present invention addressed above issues through the use of a novel localized disinfection system under its various embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

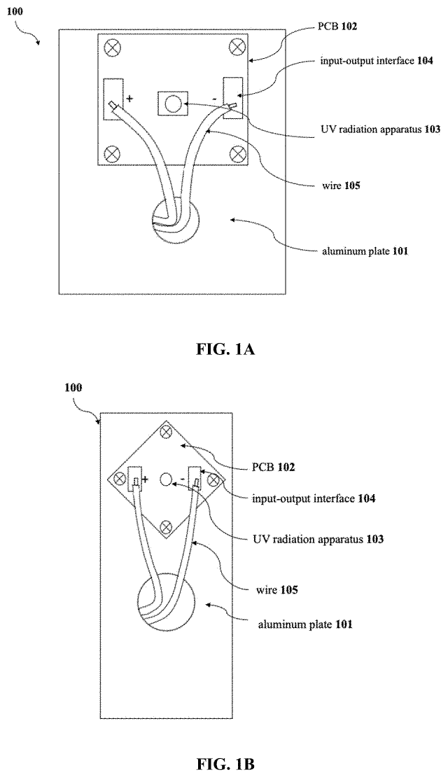

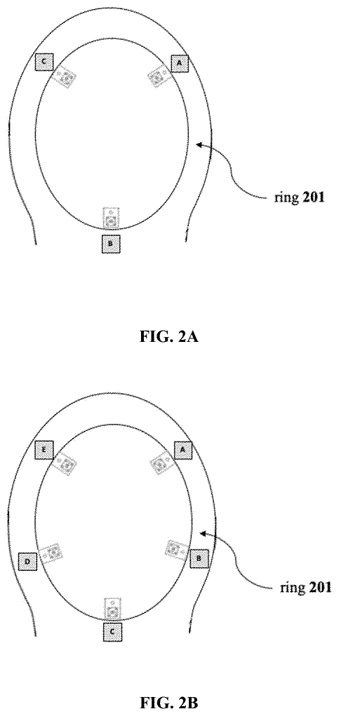

Referring to FIGS. 1A and 1B, two embodiments of disinfection devices (100) are provided as shown in FIG. 1A and FIG. 1B, respectively. Both longer and wider disinfection devices are suitable for different size of the toilet bowl. A disinfection device may be arranged to disinfect the cavity and surface of a contaminated sanitary device. The disinfection device (100) includes an aluminum plate (101), a printed circuit board (PCB) (102) fixed onto the aluminum plate (101), an ultraviolet light (UV) radiation apparatus (103) soldered on the PCB (102), and an input-output interface (104), having a positive pole and a negative pole, positioned on the UV radiation apparatus (103). The UV radiation apparatus (103) is arranged to project UV radiation towards a target area for disinfection (e.g., an opening of the sanitary device for disinfecting the air and the surface of the sanitary device above the opening). The input-output interface (104) is connected to the control circuit unit via wires (105). The disinfection device (100) is used in a disinfecting sanitary system for inactivating airborne pathogens within a sanitary device.

In one embodiment, the system further includes a control circuit unit connected with the disinfection device (100) and a detector for measuring irradiance of the disinfection device.

In various embodiments, the sanitary device is a toilet, which includes a container for receiving fluids with the airborne pathogens and a hollow member positioned in the container. The toilet further contains an opening at least partly defined by the container, and the disinfection device is positioned on the hollow member to disinfect the cavity and surface of sanitary device adjacent to the opening affected by the fluids. In addition, the disinfection device (100) could also be positioned on a cover of the toilet seat.

In one embodiment, the hollow member is an aluminum ring, and it at least partly defines the opening.

During toilet flushing, water splashing is anticipated. In order to protect the disinfection device from damage caused by ingress of the fluids, a protection member is used, which is arranged to at least partially shield the disinfection device. In one embodiment, the protection member is a transparent film with a thickness of approximately 0.1 to 0.2 mm.

In accordance with one embodiment, the UV radiation apparatus includes at least one light emitting diode (LED) arranged to irradiate within UV-C band. The at least one LEDs are distributed according to positions of a plurality of nozzles of the sanitary device arranged to create a flow of the fluids to be received in the container.

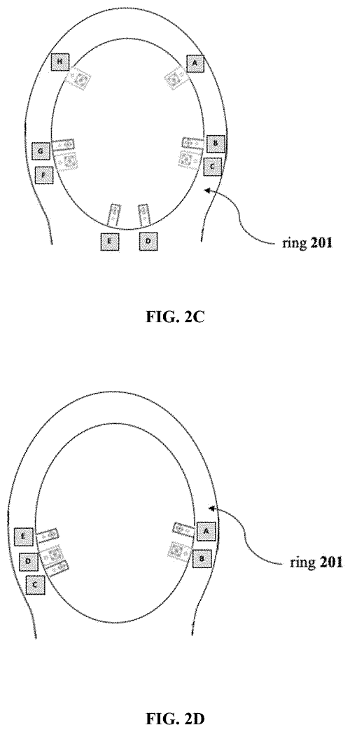

Referring to FIGS. 2A to 2D, the LEDs may have one of two distribution configurations: uniform configuration as shown FIGS. 2A-2C, and concentrated configuration as shown in FIG. 2D. More specifically, the uniform configuration comprises 3 to 8 LEDs, and the concentrated configuration comprises two-sided 5-LEDs.

In one embodiment, the plurality of disinfection devices surrounds a central axis of the hollow member at equal intervals. For 3-LEDs uniform configuration, the disinfection devices labeled as “A” to “C” are mounted on a ring (201) as shown in FIG. 2A. For 5-LEDs uniform configuration, the disinfection devices labeled as “A” to “E” are mounted on a ring (201) as shown in FIG. 2B.

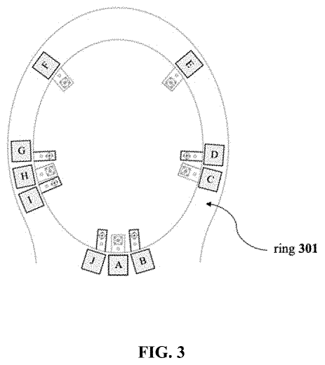

In another embodiment, an opening of the hollow member comprising at least one region, and the plurality of disinfection devices are arranged in one or more of the regions. For example, one or more of the plurality of disinfection devices are arranged at a first interval in a first region of the at least one region, one or more of the plurality of disinfection devices are arranged at a second interval in a second region of the at least one region, and one or more of the plurality of disinfection devices are arranged at a third interval in a third region of the at least one region. The first interval, the second interval and the third interval are different. For 8-LEDs uniform configuration, the disinfection devices labeled as “A” to “H” are mounted on the ring (201) as shown in FIG. 2C. In another embodiment, it is also possible to arrange one or more of the disinfection devices as shown in FIGS. 1A and 1B, and combination thereof to form a 10-LEDs uniform configuration mounted on a ring (301) as shown in FIG. 3, in which the upper region has two disinfection devices, the middle region has five disinfection devices, and the bottom region has three disinfection devices. The first, second and third interval of these three regions are different. Each of these disinfection devices (100) is connected to the control circuit unit through the input-output interface (104) via wires (105).

In the yet another embodiment, one or more of the plurality of disinfection devices are arranged in one region of the at least one region, and another region of the at least one region is not provided with the plurality of disinfection devices. For example, FIG. 2D shows the concentratedly configured disinfection devices with 5 LEDs on the ring (201). Each of these disinfection devices (100) is connected to the control circuit unit through the input-output interface (104) via wires (105).

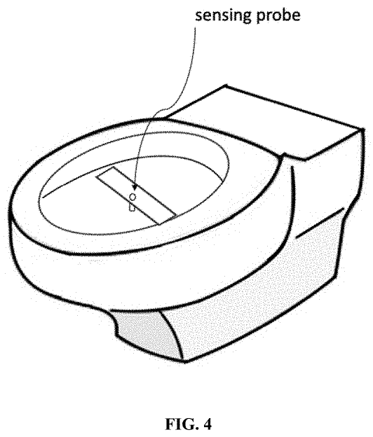

To correlate the UV irradiance level and disinfection performance, a sensing probe can serve as a detector, which can measure the total irradiance by different configurations for relative comparison. At a configuration stage, the water in the toilet bowl was drained and the sensing probe was put at a preset depth below the seating level in the middle of the bowl for measuring the total irradiance. For example, FIG. 4 shows a schematic view of a position of the sensing probe for measuring UV irradiance in the toilet bowl for different LED configurations, and such setup was used to measure the incident irradiance distribution.

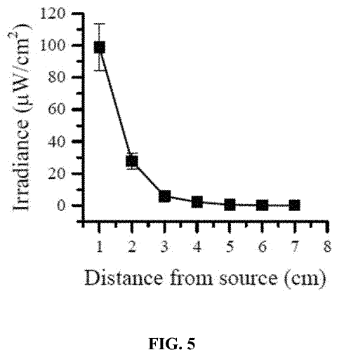

Referring to FIG. 5, the average UV-C LED irradiances at different distances were measured. Irradiance from individual UV-LEDs was measured at distances from the source up to seven centimeters, to estimate the effect of distance on irradiance changes, specifically for future modeling and estimating UV dose for the prototype unit. Here, the values reported are the means and standard deviations of the irradiance measurements taken for all the 8-LEDs used in this study. In FIG. 5, the mean LED irradiance varied from 99.02±14.72 to 0 μW/cm² when the distance increased from 1 to 7 cm. A relatively high uncertainly was found at the sample nearest to the source. At the location with such a high intensity, even a very small deviation might cause large difference in the reading. Also, it was observed that the intensity dropped very rapidly and reached close to zero when it was just 4 cm away from the LED. The decrease in irradiance with such a small distance indicates that the majority of the bacteria disinfection reported in this disclosure occurred at a short emission distance; that is, almost immediately the flushing was activated.

The results for the total irradiance for different LED configurations were reported in Table 1 below.

TABLE 1

| Configurations | Position arrangements | Total irradiance (μW/cm²) |
|---|---|---|
| 3 LEDs | A, B, C | 0.86 |
| 5 LEDs | A, B, C, D, E | 1.15 |
| 5 LEDs (two-sided) | A, B, C, D, E | 1.57 |
| 8 LEDs | A, B, C, D, E, F, G, H | 3.07 |

From Table 1, it can be seen that the total UV irradiance increased with the numbers of the LEDs tested. For the 3-LEDs, 5-LEDs, and 8-LEDs well-distributed configurations, the measured irradiances were 0.86, 1.15, and 3.07 μW/cm² respectively. Likewise, the irradiance of the 5-LEDs two-sided non-distributed configuration was 1.57 μW/cm². Due to the arrangement of the disinfection devices with LEDs, it could be observed that the total irradiance is not linearly proportional to the number of LEDs used. Besides, the two-sided 5-LEDs concentrated configuration produced a higher irradiance than the 5-LEDs uniform configuration.

It has been found that small droplets (less than 0.3 μm) would usually become airborne and could travel very long distances while coarse droplets (2-10 μm) would settle near the toilet bowl. Therefore, in the applications of the various embodiments of the present invention, the airborne pathogens are expected to be bioaerosols with a size of less than 0.3 μm, including airborne microorganisms or parasites. Once these aerosols become airborne, they can settle near the toilet bowl, and the small aerosols can stay airborne for up to hours and may lead to surface contamination, which is believed to be a major route for transmission of infective diseases.

In the applications of the various embodiments of the present invention, the target airborne microorganisms are expected to include, but not limit to, Escherichia coli, Salmonella typhimurium, Staphylococcus epidermidis, Shigella dysenteriae, Listeria monocytogenes, Clostridium difficile, and Candida albicans; and the target parasites are expected to include, but not limit to, Cryptosporidium.

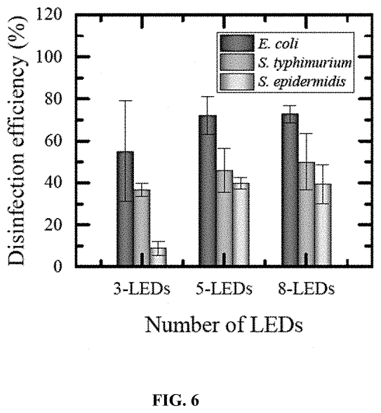

The assessment of the disinfection efficiency on the surface of a sanitary device (e.g. toilet seat) for different configurations and the bacteria under evaluation were shown collectively in FIG. 6. The estimated mean efficacies (mean±SD) with 3-LEDs, 5-LEDs and 8-LEDs were 55.17±23.89% (range 23.09-73.28%), 72.03±9.02% (range 62.86-80.89%), and 72.80±4.13% (range 69.63-77.47%) for E. coli; 36.65±2.99% (range 33.33-39.13%), 46.04±10.69% (range 35.29-56.67%), and 50.05±13.38% (range 41.30-65.45%) for S. typhimurium; 8.81±3.23% (range 5.62-12.07%), 39.63±2.72% (range 36.65-41.98%), and 39.43±9.33% (Range 30.61-49.19%) for S. epidermidis, respectively.

From these results, it was clear that the maximum surface disinfection efficacy was obtained for E. coli with 8-LEDs operational configurations. It was also noted that among the three tested bacteria, the UV irradiances had the minimum effects against S. epidermidis (as can be seen in FIG. 6).

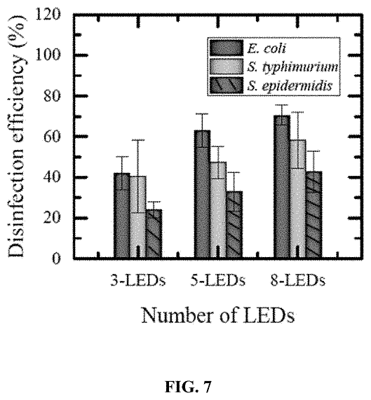

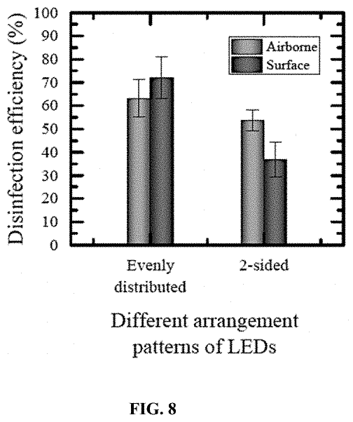

Next, the numbers of CFU at different levels (i.e. ASH, ASM and ASL) were counted and summed up. The results of the efficacy of airborne disinfection by the disinfecting sanitary system were shown in FIG. 7. The disinfection efficacies with 3-LEDs, 5-LEDs and 8-LEDs were 42.17±8.18%, 63.25±8.17% and 70.70±4.80% for E. coli; 40.40±17.90%, 47.31±8.20%, and 58.31±13.87% for S. typhimurium; 24.16±3.81%, 32.92±9.59, and 42.79±10.20% for S. epidermidis, respectively.

It one embodiment, a configuration with higher number of LEDs, specifically 10-LEDs, was also tried but no significant increase in the irradiance or disinfection efficacy was observed (data not shown). Therefore, the configuration with 8-LEDs was considered optimum for the current airborne disinfection application.

Referring to FIG. 8, the influence of UV-C LEDs configurations was further tested on the efficacy of the disinfecting sanitary system by comparing the germicidal results with the same number of LEDs but different position arrangements. Two types of UV-C LED configurations, both having 5-LEDs, one being uniform and the other one being concentrated at two opposing sides, were tested to disinfect E. coli, which was the bacteria most susceptible to UV-C LED among the three bacteria tested. For airborne disinfection, under uniform and two-sided concentrated configurations, the disinfection efficiencies were 63.25±8.17% and 53.74±4.47% respectively. Similarly, for surface disinfection, under uniform and two-sided configurations, the estimated mean efficiencies were 72.03±9.02% and 36.83±7.47% respectively. The results implied that the performances of the disinfecting sanitary system with uniform configuration were approximately 48.87% (airborne) and 15.04% (surface) higher than the two-sided concentrated configuration.

In one embodiment, the system was affixed to the sanitary device (e.g., toilet seat) and challenged by E. coli, S. typhimurium and S. epidermidis. The arrangements of the LEDs (3-LEDs, two 5-LEDs, 8-LEDs) were two-fold, uniform, and two-sided concentrated configuration. Four surface samples on the sanitary device and three air samples at different heights were collected. It was noticed that disinfection efficacy initially increased with the numbers of LEDs used, but the trends became almost insensitive with 8-LEDs for surface disinfection and slightly sensitive for airborne disinfection. For surface disinfection, the mean efficacies ranged from 55.17±23.89% to 72.80±4.13% for E. coli; 36.65±2.99% to 50.05±13.38% for S. typhimurium; and 8.81±3.23% to 39.43±9.33% for S. epidermidis. For airborne disinfection, the mean efficacies ranged from 42.17±8.18% to 70.70±4.80% for E. coli; 40.40±17.90% to 58.31±13.87% for S. typhimurium; and 24.16±3.81% to 42.79±10.20% for S. epidermidis.

### Examples

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

Test Facility:

Presented in this disclosure is an experimental chamber comprising a custom-made pre-existing toilet rig, which was equipped with an American standard wash-down type water closet (WC), one 50-liters volume water tank, and a flushometer. Also, the toilet rig was connected to a clean water supply. The UV-C LEDs had a UV-C output of less than 20 mW with the rated current of 500 mA. Each LED was soldered on a PCB and each PCB was fixed onto a small aluminum plate (12 mm×18 mm) for mounting on a tailor-made aluminum ring. The aluminum ring fitted with LEDs was then put on top of the WC for disinfection of airborne pathogens when flushing the toilet.

Different Configurations of LEDs:

Different configurations of LEDs were designed, tested and are now presented in this disclosure. First, FIGS. 2A-2C show a uniform configuration involving 3 LEDs, 5 LEDs, and 8 LEDs, respectively. Second, FIG. 2D show a concentrated configuration involving two-sided 5-LEDs. The purpose of using the uniform configuration is to achieve uniform irradiance distribution in the toilet bowl. On the contrary, the concentrated configuration is designed to mimic a non-uniform irradiance distribution scenario in the toilet bowl. Moreover, the actual design of the aluminum ring could allow mounting of at least 10 LEDs, as shown in FIG. 3.

Measurement of UV Irradiance:

To correlate the UV irradiance level and disinfection performance, the measurement of UV irradiance is required. A UV-VIS fiber-optic spectrometer (AvaSpec-ULS3648) was used to measure the irradiance. Two set of measurements were taken, one is for individual LED and the other is for different configurations. The former one was to measure irradiance for each LED to make sure the output was comparable to each other. The latter one was to measure the total irradiance for each configuration.

The disinfection of pathogens depends on the dose absorbed. It is important to measure the total irradiance by different configurations for relative comparison. In this regard, the selection of the depth of the sensing probe was arbitrary. The water in the toilet bowl was drained and the probe was put at a preset depth below the seating level in the middle of the bowl for measuring the total irradiance. For example, FIG. 4 showed a schematic view of a position of the sensing probe for measuring UV irradiance in the toilet bowl for different LED configurations, and such setup was used to measure the incident irradiance distribution.

Microorganism Selection:

The criteria for the selection of micro-organisms include biosafety issues and pathogenic properties. Presented in this disclosure are three selected species of pathogenic bacteria, including Escherichia coli (E. coli) (ATCC #10536), Salmonella typhimurium (S. typhimurium) (ATCC #53648), and Staphylococcus epidermidis (S. epidermidis) (ATCC #12228). These bacteria have been previously used as nonpathogenic surrogate species in other bioaerosol and surface contamination studies. Also, the procedures for the preparation of these bacteria have been published.

Experimental Procedure:

Prior to seeding the toilet bowl with bacteria, the toilet bowl and the cistern were thoroughly cleaned with 100 ml of commercially available Clorox (chlorine) bleach and a toilet brush, and then flushed three times to completely remove residues of the cleaning compound and any micro-organisms present in flushing water. A solution of 12 ml of sodium thiosulphate was then added to inactivate any bleach chemicals present in the water. Finally, water was again used to wash the bowl and cistern in the same manner as previously described. The water used for this disclosure was public utility water and had been filtered to remove suspended solids or microbes. This cleaning process was repeated before each experiment. After thoroughly cleaning the system, the tank was filled with water. During the cleaning process, the air in the chamber was simultaneously disinfected using an upper-room ultraviolet germicidal irradiation (UR-UVGI) system which was installed at the upper part of one of the chamber walls. The UR-UVGI system was turned off when the cleaning of the toilet bowl and cistern was completed.

At the end of the cleaning task, the LEDs were fixed to the rim of the WC Subsequently, three air sampling components were installed at carefully selected locations to mimic the inhalation of different toilet users and categorized as low-level air samples (ASL) for seat level initial upsurge of aerosol from the flushed toilet bowl, middle-level air samples (ASM) for children's breathing zone, and high-level air samples (ASH) for adults' breathing zone. The vertical distances from the ground floor level to the ASL, ASM and ASH levels were 0.4 m, 0.9 m, and 1.3 m respectively.

To simplify the collection of air samples at the three levels mentioned, three copper sampling tubes were used. Each copper tube, 0.012 m in diameter and 1 m in length, was connected to a cast acrylic sheet squared box (0.15 m×0.15 m×0.15 m) at one end. The cast acrylic sheet squared box was then connected to the impactor and the three air sampling manifolds were carefully adjusted to align the other ends of the copper tubes to the center of the toilet bowl. The air samples were transferred to the agar plates through the copper tubes and the cast acrylic sheet square box.

Also, to measure bioaerosols deposited onto the toilet seat, four nutrient agar-filled plates with lids were set out in predetermined positions near the edges of the toilet seat, labeled S1 (front side), S2 (right side), S3 (left side), and S4 (back side).

Thereafter, a 250-mL of bacteria solution was poured from a vial into the toilet bowl (seeding). Having seeded the toilet bowl, lids of the agar plates for surface sample collection were opened and the LEDs were activated. The door of the toilet chamber was closed, and the flush was triggered. Since a high priority was given to safety, no one was allowed inside the chamber during the experiments. To activate the flushing, a long string was attached to the flush lever, to allow the toilet to be flushed from outside the test room. At the time the toilet was flushed to generate airborne microorganism emission, the three single-stage Anderson biological impactors were also run for 1 min with calibrated vacuum pumps operated at a flow rate of 28.3 L/min. The vacuum pump drew air samples from the experimental toilet facility into the inlet of the impactor and then aims the particle-laden airstream at the nutrient-filled medium spread on the agar plate. After allowing an additional 15 min for droplets emitted from the one-time toilet flush to settle onto the agar plates, the door was unsealed to collect all plates. After a collection cycle, all of the seven agar plates (three air and four surface samples) were then immediately incubated at 37° C. overnight and colony-forming units (CFUs) were counted.

Control experiments were also conducted in the same environmental conditions without exposure to UV-C LED irradiation for equal time points and on the same day as the treatment experiment. During the experiments, the environmental conditions such as relative humidity and temperature were closely monitored and were kept the same between control and treatment.

Data Interpretation and Statistical Analysis:

Each inactivation experiment was repeated in triplicate. Each data bar represents the arithmetic mean of the three replicates and the standard deviation of the three trials was used as the error bar. The disinfection efficiency (η) was estimated using the following equation:

$$\overline{η} = [ 1 - ( {\sum }_{i = 1}^{n} \frac{{CFU}_{UV - on , i}}{{CFU}_{UV - off , i}} ) ] \times 100 \% ,$$

where CFUᵤᵥ₋ₒₙ and CFU_{uv-off} are the colony-forming units with and without LED exposure, respectively at the same time point, n represents the number of samples taken (in this disclosure, 3 for airborne and 4 for surface samples). The p-value was used to determine the statistical significance of the observed differences. A positive hole correction factor was applied to the raw CFU counts on the Petri dish after appropriate incubation.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

This study is one of the first to demonstrate an intervention technology for inactivating enteropathogenic bacteria. No other new interventions were implemented during the study period, suggesting that the decrease in the incidence of flushing-generated toilet pathogens was due solely to the usage of localized UV-C LEDs for disinfection.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

1. A disinfecting sanitary system for inactivating airborne pathogens within a sanitary device comprising
a plurality of disinfection devices mounted on a hollow member of the sanitary device, and each of the disinfection devices is configured to emit a beam for disinfection; and
a control circuit coupled to the plurality of disinfection devices,
wherein each of the plurality of disinfection devices includes an ultraviolet light (UV) radiation apparatus configured to project UV radiation towards a target area for disinfection, and
wherein the control circuit controls the plurality of disinfection devices to project the UV radiation.

2. The disinfecting sanitary system according to claim 1, wherein the plurality of disinfection devices further comprise an aluminum plate and a printed circuit board (PCB) fixed onto the aluminum plate.

3. The disinfecting sanitary system according to claim 2, wherein the plurality of disinfection devices further comprises an input-output interface having a positive pole and a negative pole communicating with the UV radiation apparatus, wherein the input-output interface communicates with the control circuit.

4. The disinfecting sanitary system according to claim 1, wherein the disinfecting sanitary system further comprises a protection member wrapping the UV radiation apparatus.

5. The disinfecting sanitary system according to claim 4, wherein the protection member is a transparent film with an approximate thickness in the range of 0.1 to 0.2 mm.

6. The disinfecting sanitary system according to claim 1, wherein the UV radiation apparatus comprises at least one light emitting diode (LED) distributed according to positions of a plurality of nozzles of the sanitary device, and the at least one LED is/are arranged to irradiate within UV-C band.

7. The disinfecting sanitary system according to claim 6, wherein the peak wavelength of the at least one LED is in the range of 100 nm to 280 nm.

8. The disinfecting sanitary system according to claim 1, wherein the plurality of disinfection devices surrounds a central axis of the hollow member at equal intervals.

9. The disinfecting sanitary system according to claim 1, wherein an opening of the hollow member comprising at least one region, and the plurality of disinfection devices are arranged in one or more of the regions.

10. The disinfecting sanitary system according to claim 9, wherein one or more of the plurality of disinfection devices are arranged at a first interval in a first region of the at least one region, wherein one or more of the plurality of disinfection devices are arranged at a second interval in a second region of the at least one region, wherein one or more of the plurality of disinfection devices are arranged at a third interval in a third region of the at least one region, and wherein the first interval, the second interval and the third interval are different.

11. The disinfecting sanitary system according to claim 9, wherein one or more of the plurality of disinfection devices are arranged in one region of the at least one region, and another region of the at least one region is not provided with the plurality of disinfection devices.

12. The disinfecting sanitary system according to claim 1, wherein the disinfecting sanitary system inactivates the airborne pathogens within a vertical distance of 0.4 m to 1.3 m from a ground floor level.

13. The disinfecting sanitary system according to claim 1, wherein the airborne pathogens are bioaerosols with a size of less than 0.3 μm, and the bioaerosols comprise airborne microorganisms or parasites.

14. The disinfecting sanitary system according to claim 13, wherein the microorganisms are select from the group consisting of Escherichia coli, Salmonella typhimurium, Staphylococcus epidermidis, Shigella dysenteriae, Listeria monocytogenes, Clostridium difficile, and Candida albicans.

15. The disinfecting sanitary system according to claim 13, wherein the parasites comprise Cryptosporidium.

16. A sanitary device for inactivating airborne pathogens comprising the disinfecting sanitary system of claim 1.

17. The disinfecting sanitary system according to claim 16, wherein the sanitary device comprises a container for receiving fluids and the airborne pathogens, and a hollow member positioned on the container.

18. The disinfecting sanitary system according to claim 17, wherein the sanitary device further comprises an opening at least partly defined by the container.

19. The disinfecting sanitary system according to claim 18, wherein the hollow member is an aluminum ring, and wherein the hollow member at least partly defining the opening.
